(19) United States
(12) Patent Application Publication
(10) Pub. No.: US 2012/0330287 A1
(43) Pub. Date: Dec. 27, 2012

(54) SURGICAL INSTRUMENT

(75) Inventor: Jin Whan Yim, Goyang-si (KR)

(73) Assignee: ADEN LIMITED, Goyang-si (KR)

(21) Appl. No.: 13/581,520

(22) PCT Filed: Mar. 15, 2010
(86) PCT No.: PCT/KR2010/001595

§ 371 Date: Aug. 28, 2012

(51) Int. Cl. A61B 17/00

(52) U.S. Cl. 606/1

(57) ABSTRACT

Provided is a surgical instrument, including: a shaft; a wrist member including a guide member, a wrist body connected to the guide member and formed with a plurality of installation holes, and a plurality of protruding members each disposed to face the wrist body and having an insertion groove, and connected to the shaft; a plurality of effectors each connected to the wrist member by a pin; a support driving unit having first through fourth rotation members inserted into pins that are mounted on the wrist member, respectively, and each first and second wires connected to the plurality of effectors and the first through fourth rotation members, respectively; a main driving unit connected to the support driving unit by wire and having at least one driving unit. The main driving unit enables the entire wrist member to perform a yaw motion by rotating the entire wrist member, and enables the plurality of effectors to perform a pitch motion by simultaneously rotating the plurality of effectors into a single direction, and enables the plurality of effectors to perform a forceps motion of rotating into a direction increasing or decreasing a distance between the plurality of effectors.

### BACKGROUND OF THE INVENTION

1. Field of the Invention

The present invention relates to a surgical instrument, and more particularly, to a surgical instrument that may decrease the overall diameter of a surgical instrument by forming a wrist joint on a rear idler pulley between a front idler pulley and the rear idler pulley and thereby making it possible to decrease a diameter of the front idler pulley.

2. Description of the Related Art

To reduce a period of recuperation of a patient by minimizing an incision during an operation, a surgical instrument for surgery department may be applied to a laparoscopic surgery or a robot surgery. The surgical instrument for surgery department may be applied to the laparoscopic surgery or the robot surgery and hereinafter, may be simply referred to as a surgical instrument.

The conventional art associated with the surgical instrument is already disclosed in the U.S. Pat. No. 5,792,135 that is registered in Aug. 6, 1998, and applied by the Applicant Institute Surgical. Inc. Hereinafter, the surgical instrument disclosed in the U.S. Pat. No. 5,792,135 will be described with reference to FIG. 1.

As shown in FIG. 1, the conventional surgical instrument may include a wrist-like mechanism 1100 and a shaft 1200.

The wrist-like mechanism 1100 is connected to the shaft 1200 and includes a wrist member 1110, a plurality of capstans 1120, an end effector 1130, a front idler pulley 1140, a rear idler pulley 1150, and a plurality of wire loops 1160 and 1170. Even though the end effector 1130, the front idler pulley 1140, and the rear idler pulley 1150 are illustrated to be single, respectively, in FIG. 1, the conventional surgical instrument includes a plurality of end effectors 1130, a plurality of idler pulleys 1140, and a plurality of rear idler pulleys 1150. A rotation shaft of the front idler pulley 1140 forms a wrist joint 1140a.

A diameter of the conventional surgical instrument is determined by a diameter d1 of a plurality of protruding members 1110a formed on the wrist member 1110, and a maximum value of a diameter d2 of the capstan 1120 is constrained by the diameter d1 of the surgical instrument. The diameter d1 of the protruding member 1110 is determined based on a radius r1 of the front idler pulley 1140 that forms the wrist joint 1140 mounted to be close to the protruding member 1110a. A distance g1 between the plurality of capstans 1120 is determined based on the radius r1 of the front idler pulley 1140.

In the conventional surgical instrument, when the radius r1 of the front idler pulley 1140 is great, the distance g1 between the plurality of capstans 1120 may also increase. When the distance g1 between the plurality of capstans 1120 increases, the diameter d2 of the capstan 1120 may decreases, thereby requiring a great torque.

Also, operation power of the surgical instrument is in proportion to tension applied to the plurality of wire loops 1160 and 1170. Rotary force of the end effectors 1130 is determined based on the tension applied to the plurality of wire loops 1160 and 1170 and the radius of the wrist joint 1140a. As shown in FIG. 1, in the conventional surgical instrument, the wrist joint 1140a is positioned on the front idler pulley 1140 and thus, the rotary force of the end effectors 1130 may be determined based on the tension applied to the plurality of wire loops 1160 and 1170 and the radius r1 of the front idler pulley 1140.

In the conventional surgical instrument, the wrist joint 1140a is formed on the front idler pulley 1140. Therefore, when decreasing the radius r1 of the front idler pulley 1140, tension to be applied to the plurality of wire loops 1160 and 1170 may increase and durability of the plurality of wire loops 1160 and 1170 may be degraded. When the durability of the plurality of wire loops 1160 and 1170 is degraded, reliability of the surgical instrument may also be degraded.

Also, when it is impossible to decrease the radius r1 of the front idler pulley 1140, the distance g1 between the plurality of capstans 1120 may increase and thus, it may be impossible to increase the diameter d2 of the capstan 1120. Due to the small diameter d2 of the capstan, the conventional surgical instrument requires relatively great torque in order to drive the end effector 1130 and thus, may not be readily operated.

### SUMMARY OF THE INVENTION

As described above, the conventional surgical instrument may not decrease a radius of a front idler pulley since a wrist joint is formed on the front idler pulley. Accordingly, the conventional surgical instrument has a problem that it is impossible to reduce the overall diameter of the surgical instrument.

To overcome the above problem, an aspect of the present invention provides a surgical instrument that may decrease a surgery portion of a patient to be as small as possible by forming a wrist joint on a rear idler pulley between a front idler pulley and the rear idler pulley and reducing a diameter of the front idler pulley, thereby leading to reducing the overall diameter of the surgical instrument.

Another aspect of the present invention also provides a surgical instrument that may minutely control an end effector by applying small operation power to a wire since a diameter of a capstan may be formed to be large by reducing the diameter of the front idler pulley.

Another aspect of the present invention also provides a surgical instrument that may enhance reliability of a surgical instrument by decreasing operation power to be applied to a wire and thereby enhancing durability of the wire.

According to a first embodiment of the present invention, there is provided a surgical instrument including: a shaft; a wrist member including a guide member, a wrist body connected to the guide member and formed with a plurality of installation holes, and a plurality of protruding members each disposed to face the wrist body and having an insertion groove, and connected to the shaft; a plurality of effectors connected to the wrist member by a pin; a support driving unit having first through fourth rotation members inserted into pins that are mounted on the wrist member, respectively, and first and second wires connected to the plurality of effectors and the first through fourth rotation members, respectively; and a main driving unit including first through third main driving units. The main driving unit enables the entire wrist member to perform a yaw motion by rotating the entire wrist member, and enables the plurality of effectors to perform a pitch motion by simultaneously rotating the plurality of effectors into a single direction, and enables the plurality of effectors to perform a forceps motion of rotating into a direction increasing or decreasing a distance between the plurality of effectors.

According to a second embodiment of the present invention, there is provided a surgical instrument including: a shaft; a wrist member including a guide member, a wrist body connected to the guide member and formed with a plurality of installation holes, and a plurality of protruding members each disposed to face the wrist body and having an insertion groove, and connected to an effector; a plurality of effectors connected to the wrist member by a pin; a support driving unit having first through fourth rotation members inserted into pins that are mounted on the wrist member, respectively, and first and second wires sequentially connected to the plurality of effectors and the first through fourth rotation members, respectively; and a main driving unit connected to the support driving unit and mounted to be twisted with respect to the plurality of effectors. The main driving unit enables the entire wrist member to perform a yaw motion by rotating the entire wrist member, and enables the plurality of effectors to perform a pitch motion by simultaneously rotating the plurality of effectors into a single direction, and enables the plurality of effectors to perform a forceps motion of rotating into a direction increasing or decreasing a distance between the plurality of effectors.

### BRIEF DESCRIPTION OF THE DRAWINGS

The above and/or other aspects of the present invention will become apparent and more readily appreciated from the following description of the exemplary embodiments, taken in conjunction with the accompanying drawings in which:

FIG. 1 is a diagram illustrating a preparing a preparation method of a metal oxide doped monolith carbon aerogel for a high capacitance capacitor according to the present invention;

FIG. 2 is a schematic perspective view illustrating a surgical instrument according to a first embodiment of the present invention;

FIG. 3 is a perspective view illustrating a portion of the surgical instrument being exploded of FIG. 2;

FIG. 4 is an exploded perspective view illustrating a driving unit of FIG. 3;

FIG. 5 is a view illustrating another embodiment of a cable driving unit of FIG. 3;

FIGS. 6 and 7 are views to describe an operation state of the surgical instrument;

FIG. 8 is a schematic perspective view illustrating a surgical instrument according to a second embodiment of the present invention;

FIG. 9 is an exploded perspective view illustrating a portion of the surgical instrument of FIG. 8;

FIG. 10 is a view illustrating another embodiment of a cable driving unit of FIG. 9; and

FIG. 11 is a cross-sectional view to describe a characteristic of a support driving unit of the present invention.

### DETAILED DESCRIPTION OF THE INVENTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

Reference will now be made in detail to embodiments of the present invention, examples of which are illustrated in the accompanying drawings, wherein like reference numerals refer to the like elements throughout. Hereinafter, a surgical instrument according to a first embodiment of the present invention will be described with reference to the accompanying drawings.

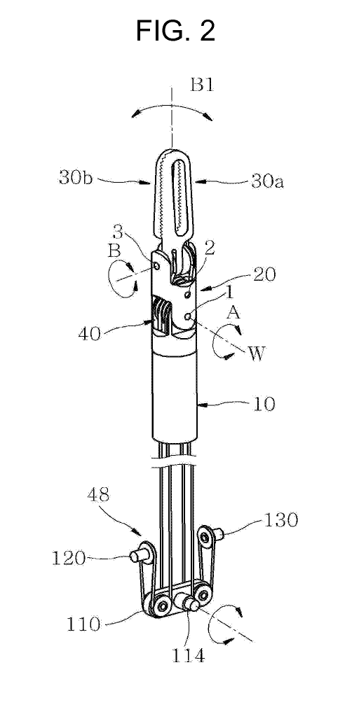

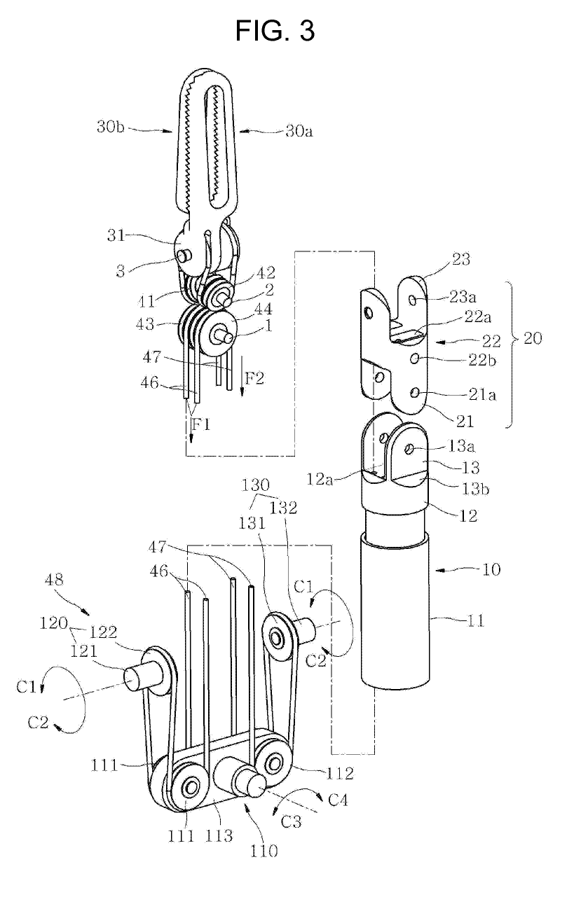

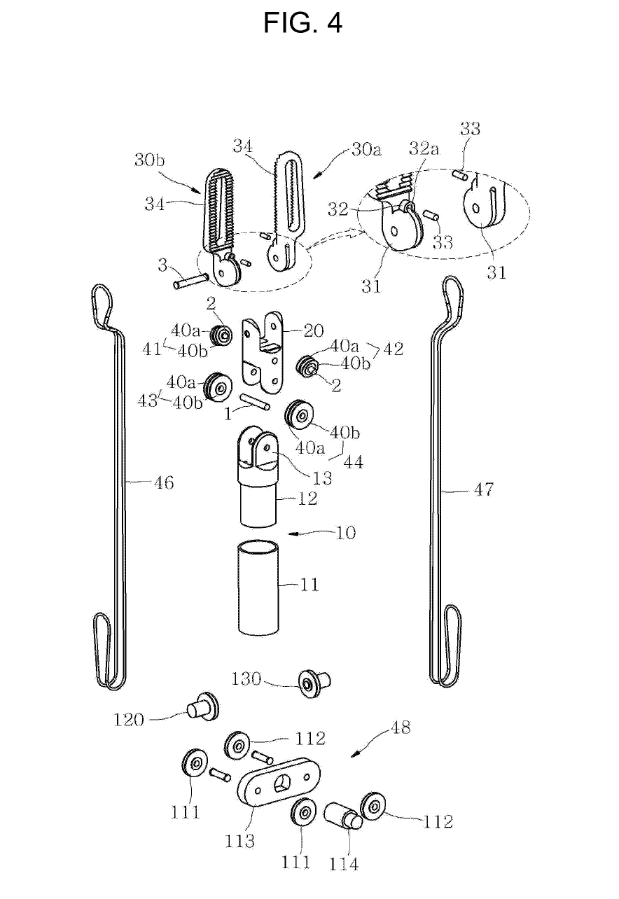

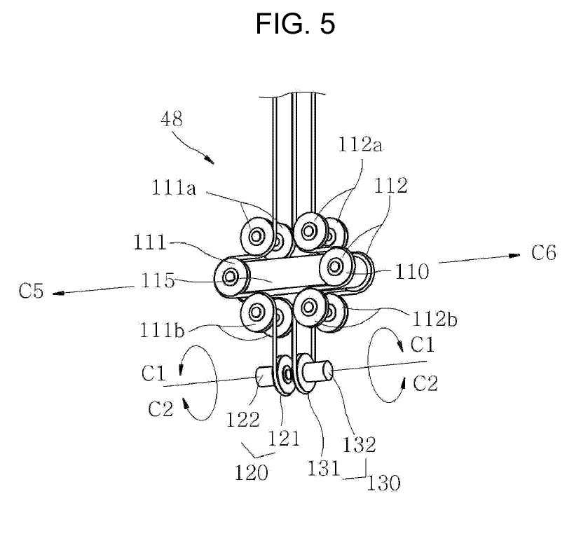

FIG. 2 is a perspective view illustrating an assembly state of a surgical instrument of the present invention, FIG. 3 is a perspective view illustrating a portion of the surgical instrument being exploded of FIG. 2, FIG. 4 is an exploded perspective view illustrating a driving unit of FIG. 3, and FIG. 5 is a view illustrating another embodiment of a driving unit of FIG. 3.

The surgical instrument according to the first embodiment of the present invention includes a shaft 10, a wrist member 20, a plurality of effectors 30a and 30b, a support driving unit 40, and a main driving unit 48 including first through third main driving unit 110,120, and 130.

In the surgical instrument of the present invention, the shaft 10 is configured to support the surgical instrument overall, and the wrist member 20 is connected to the shaft 10 by a first pin 1, and a plurality of second pins 2 is inserted into the wrist member 20. Each of the plurality of effectors 30a and 30b is connected to the wrist member 20 by a third pin 3. The support driving unit 40 and the first through third main driving units 110,120, and 130 enable the wrist member 20 to perform a yaw motion by rotating the wrist member 20, and enable the plurality of effectors 30a and 30b to perform a pitch motion of moving into a single direction different from a direction of the yaw motion by simultaneously rotating the plurality of effectors 30a and 30b, and enable the effectors 30a and 30b to perform a forceps motion of rotating into a direction increasing or decreasing a distance between the plurality of effectors 30a and 30b.

As shown in FIGS. 3 and 4, the shaft 10 includes a hollow member 11, a support member 12, and a plurality of protruding members 13. The hollow member 11 is configured to support the surgical instrument overall, and the support member 12 is inserted into the hollow member 11, and a plurality of guide holes 12a is formed in the support member 12. The plurality of protruding members 13 is extended along the support member 12, and a pair of protruding members 13 are formed to face each other. An insertion hole 13a is formed in each of the protruding members 13 to be inserted with the first pin 1. An inclined surface 13a is formed between the support member 12 and the protruding member 13 to thereby function to guide the yaw motion of the wrist member 20.

The wrist member 20 includes a plurality of guide members 21, a wrist body 22, and a plurality of protruding members 23.

The plurality of guide members 21 is disposed below the wrist body 22 to face each other, and an insertion hole 21a is formed in each guide member 21 to be inserted with the first pin 1. The plurality of guide members 21 is disposed below the wrist body 22 to face each other and thus, a pair of third rotation members 43 and a pair of fourth rotation members 44 of the support driving unit 40 are inserted by the first pin 1 to be disposed between the plurality of guide members 21. The plurality of guide members 21 is inserted into the shaft 10 by making the plurality of protruding members 13 be positioned inside the shaft 10.

The wrist body 22 is extended from the plurality of guide members 21, is formed with a plurality of installation holes 22a, and is connected to the installation holes 22a. A plurality of insertion holes 22b is formed in the wrist body to be inserted with the second pin 2. The plurality of protruding members 23 is disposed above the wrist body 22 to face each other, and a plurality of insertion holes 23a is formed in the protruding members 23 to be inserted with the third pin 3.

Each of the plurality of effectors 30a and 30b includes a capstan 31, a protruding member 32, a fixing member 33, and an end effector 34. The capstan 31 is inserted by the third pin 3, the protruding member 32 is integrally formed with the capstan 31, and an insertion hole 32a is formed therein. The fixing member 33 is inserted into the insertion hole 32a of the protruding member 32, and the end effector 34 is formed on the capstan 31 to perform a pitch motion by rotation of the capstan 31.

The support driving unit 40 includes a first rotation member 41, a second rotation member 42, a third rotation member 43, a fourth rotation member 44, a first wire 46, and a second wire 47.

The first rotation member 41 is inserted by one of a plurality of second pins 2 mounted on the wrist member 20, and the second rotation member 42 is inserted by another one of a plurality of second pins 2 mounted on the wrist member 20. Such first rotation member 41 and the second rotation member 42 are mounted to be inclined on the installation hole 22a formed in the wrist body 22. The third rotation member 43 is inserted by the first pin 1 to be positioned on one side of the wrist member 20, and the fourth rotation member 44 is inserted by the first pin 1 to be positioned on another side of the wrist member 20.

Each of the first through fourth rotation members 41, 42, 43, and 44 includes a pair of idler pulleys 40a and 40b that are mounted to be adjacent to each other. The pair of idler pulleys 40a and 40b mounted to each of the first and second rotation members 41 and 42 correspond to front idler pulleys, and a pair of idler pulleys mounted to each of the third and fourth rotation members 43 and 44 correspond to rear idler pulleys. Also, when the third rotation member 43 and the fourth rotation member 44 rotate whereby the wrist member 20 performs a yaw motion based on the first pin 1, the first rotation member 41 and the second rotation member 42 perform only guide functionality in the case of adjusting a position of the first wire 46 or the second wire 47.

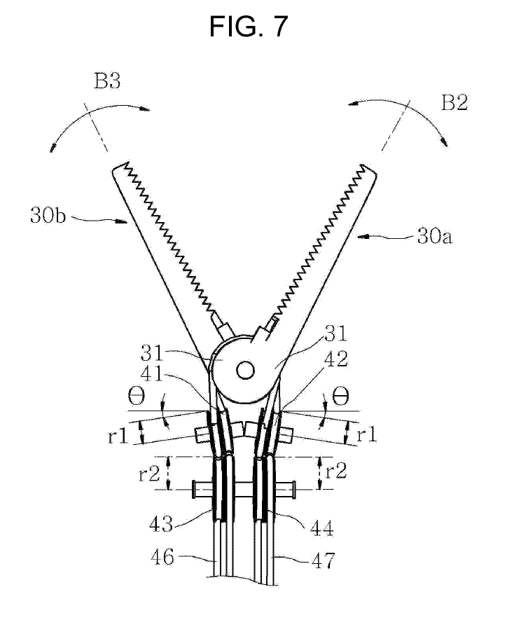

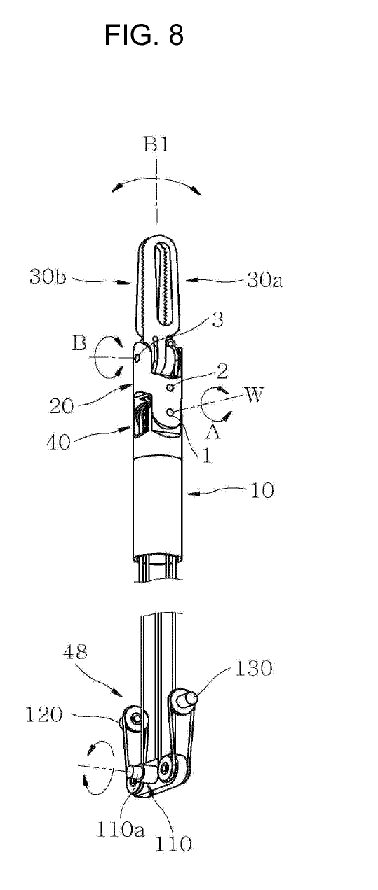

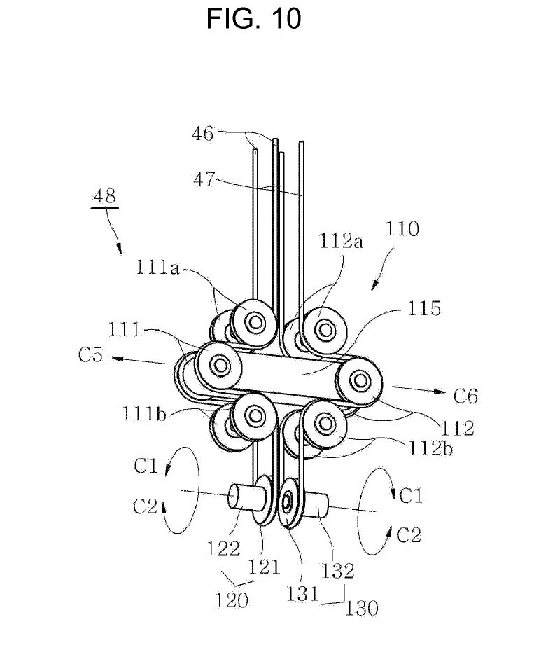

Accordingly, a radius r2 (see FIGS. 7 and 10) of the first rotation member 41 and the second rotation member 42 is formed to be less than a radius r1 (see FIG. 4) of the third rotation member 43 and the fourth rotation member 44, and the radius r2 of the first rotation member 41 and the second rotation member 42 is formed to be 0.3 to 0.9 folds of the radius r1 of the third rotation member 43 and the fourth rotation member 44. Accordingly, the diameter of the first and second rotation members 41 and 42 may be formed to be small compared to the diameter of the third and fourth rotation members 43 and 44.

The first wire 46 is connected to one idler pulley 40a provided to each of the first through fourth rotation member 41, 42, 43, and 44 and one capstan 31 among the plurality of effectors 30a and 30b to thereby rotate the corresponding rotation member. Such first wire 46 is connected to one idler pulley 40a provided to each of the first through fourth rotation members 41, 42, 43, and 44 to be crossed in an “S” shape or an inverse “S” shape.

The second wire 47 is connected to another one idler pulley 40b provided to each of the first through fourth rotation member 41, 42, 43, and 44, and another capstan 31 among the plurality of effectors 30a and 30b to thereby rotate the corresponding rotation member. Such second wire 47 is connected to the other one idler pulley 40a provided to each of the first through fourth rotation members 41, 42, 43, and 44 to be crossed in the “S” shape or the inverse “S” shape.

In the case of connecting, to the capstan 31, each of the first wire 46 and the second wire 47 that are connected to be crossed, the corresponding wire is fixed to the capstan 31 by the fixing member 33 of the effectors 30a and 30b and is inserted into the plurality of guide holes 12a formed in the support member 12 of the shaft 10 to be guided and thereby be moved.

The main driving unit 48 is mounted to be spaced apart from the shaft 10, and is connected to the first wire 46 and the second wire 47 to thereby adjust a position of the first wire 46 and the second wire 47. The main driving unit 48 includes the first main driving unit 110, the second main driving unit 120, and the third main driving unit 130.

The first main driving unit 110 is mounted to be spaced apart from the shaft 10, and is connected to the first wire 46 and the second wire 47 to thereby enable the wrist member 20 to perform the yaw motion based on the first pin 1. A pair of first rotation guide members 111, a pair of second rotation guide members 112, and a seesaw motion member 113 are further mounted to the first main driving unit 110.

The pair of first rotation guide members 111 are connected to the first wire 46, and are mounted on both sides of the seesaw motion member 113, respectively. The pair of rotation guide members 112 are connected to the second wire 47, and are mounted on both sides of the seesaw motion member 113, respectively.

The seesaw motion member 113 is mounted with the pair of first rotation guide members 111 and the pair of rotation guide members on both sides to perform a seesaw motion by rotation of the first main driving unit 110 and thereby adjust a position of the first wire 46 or the second wire 47. A shaft direction of the first main driving unit 114 is configured to be identical to a shaft direction of the first pin 1 to be combined with the shaft 10 and the wrist member 20 as shown in FIG. 2.

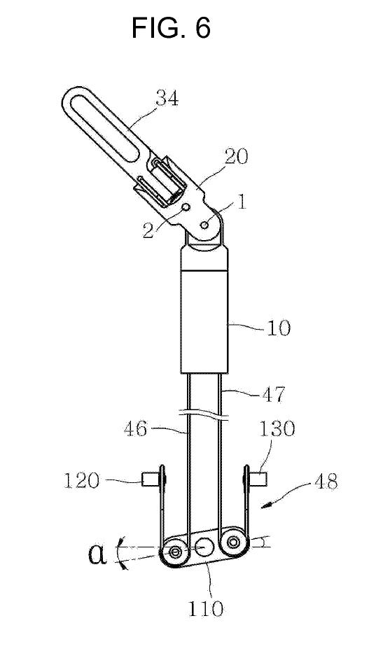

The first main driving unit 110 rotates manually or by a driving source to thereby rotate the seesaw motion member 113 into indicator directions C3 and C4 and thereby enables the seesaw motion member 113 to rotate by an angle of rotation α (see FIG. 6). When the seesaw motion member 113 rotates by the angle of rotation α and thereby performs the seesaw motion, the first wire 46 and the second wire 47 may be moved into an indicator direction F1 whereby a position may be adjusted. When the position of the first wire 46 and the second wire 47 is adjusted by the seesaw motion of the seesaw motion member 113, the wrist member 20 may perform the yaw motion based on the first pin 1 by the adjusted position. Due to the above position adjustment, when pulling one of the first wire 46 and the second wire 47 in a state where the first wire 46 or the second wire 47 forms a loop, another one thereof may be pulled into an opposite direction. An idler pulley is applied to each of the pair of first rotation guide members 111 and the pair of second rotation guide member 112.

As another embodiment of the aforementioned main driving unit 48, as shown in FIG. 5, the main driving unit 48 includes a pair of first rotation guide member 111, a pair of second rotation guide members 112, a pair of first support rotation guide members 111a, a pair of second support rotation members 111b, a pair of third support rotation guide members 112a, a pair of fourth support rotation guide members 112b, and a linear motion member 115.

The pair of first rotation guide members 111 are connected to the first wire 46, and the pair of second rotation guide members 112 are connected to the second wire 47. The pair of first support rotation guide members 111a are mounted on front of the pair of first rotation guide members 111, and are connected to the first wire 46. The pair of second support rotation guide members 111b are mounted on rear of the pair of first rotation guide members 111, and are connected to the first wire 46. The pair of third support rotation guide members 112a are mounted on front of the pair of second rotation guide members 112, and are connected to the second wire 47. The pair of four support rotation guide members 112b are mounted on rear of the pair of second rotation guide members 112 and are connected to the second wire 47. Each of the pair of first rotation guide members 111, the pair of second rotation guide members 112, the pair of first support rotation guide members 111a, the pair of second support rotation guide members 111b, the pair of third rotation support guide members 112a, and the pair of fourth rotation support guide members 112b is mounted to the linear motion member 115, and is applied with an idler pulley.

The linear motion member 115 is mounted with the pair of first rotation guide members 111 and the pair of second rotation guide members 112 on both sides, respectively, and performs a linear motion to thereby adjust a position of the first wire 46 or the second wire 47. The linear motion member 115 is connected to a linear motion mechanism (not shown) to perform the linear motion into indicator directions C5 and C6 by way of the linear motion mechanism. When the linear motion member 115 performs the linear motion into the indicator directors C5 and C6, a displacement corresponding to the linear motion may occur in the position of the first wire 46 or the second wire 47. Due to the displacement of wire position, the wrist member 20 may perform the yaw motion based on the first pin 1.

The second main driving unit 120 is mounted to be spaced apart from the first main driving unit 110, and is connected to the first wire 46 through the first main driving unit 110 to thereby enable one of the plurality of effectors 30a and 30b to perform a pitch motion based on the third pin 3. For example, when the second main driving unit 120 rotates into the indicator direction C1 or C2 whereby the position of the first wire 46 is moved and thereby changed into an indicator direction F2, one end effector 34 among the plurality of effectors 30a and 30b may perform the pitch motion by the changed position displacement.

The third main driving unit 130 is disposed to face the second main driving unit 120, and is connected to the second wire 47 through the first main driving unit 110 to thereby enable another one of the plurality of effectors 30a and 30b to perform the pitch motion based on the third pin 3. For example, when the third main driving unit 130 rotates into the indicator direction C1 or C2, the position of the second wire 46 may be changed and another end effector 34 among the plurality of effectors 30a and 30b may perform the pitch motion based on the third pin 3.

Along the rotation direction of the second main driving unit 120 and the third main driving unit 130, the plurality of effectors 30a and 30b may individually rotate to perform the forceps motion or may simultaneously rotate to perform the pitch motion. For example, when the second main driving unit 120 rotates into the indicator direction C1 and the third main driving unit 130 rotates into the indicator direction C2 different from C1, the end effectors 34 of the plurality of effectors 30a and 30b may individually perform the pitch motion and thus, may perform the forceps motion of operating into a direction increasing or decreasing a distance between the second main driving unit 120 and the third main driving unit 130. On the contrary, when the second main driving unit 120 and the third main driving unit 130 simultaneously rotate into the indicator direction C1 or C2, the end effectors 34 provided to the plurality of effectors 30a and 30b may simultaneously perform the pitch motion.

The second main driving unit 120 and the third main driving unit 130 that enable the end effectors 34 to perform the pitch motion include second rotation shafts 122 and 132, and pulleys 121 and 131, respectively. Each of the second rotation shafts 122 and 132 is mounted on front or rear of the first main driving unit 110, and is connected to a driving source (not shown). The second rotation shafts 122 and 132 connected to the driving source rotate into the indicator directions C1 and C2 by operation of the driving source. The pulleys 121 and 131 are connected to the second rotation shafts 122 and 132, and rotate by rotation of the second rotation shafts 122 and 132, respectively, to thereby adjust a position of the first wire 46 or the second wire 47. That is, the pulleys 121 and 131 rotating by the second rotation shafts 122 and 132 that rotate by the driving source may adjust the position of the first wire 46 or the second wire 47. Such pulleys 121 and 131 are mounted to cross the pair of first rotation guide members 111 and the pair of second rotation guide members 112.

Hereinafter, a surgical instrument according to a second embodiment of the present invention will be described with reference to the accompanying drawings.

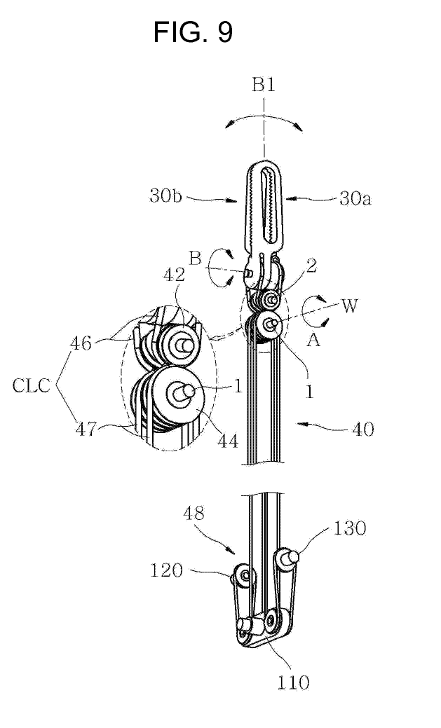

FIG. 9 is a perspective view illustrating an assembly state of a surgical instrument according to the second embodiment of the present invention, and FIG. 10 is a view illustrating another embodiment of a driving unit of FIG. 9.

The surgical instrument according to the second embodiment of the present invention includes a shaft 10, a wrist member 20, a plurality of effectors 30a and 30b, a support driving unit 40, and a main driving unit 48. Here, the wrist member 20 and the plurality of effectors 30a and 30b may be configured to be similar to the first embodiment of the present invention and thus, a further detailed description related thereto will be omitted here.

The support driving unit 40 according to the second embodiment of the present invention includes a first rotation member 41, a second rotation member 42, a third rotation member 43, a fourth rotation member 44, a first wire 46, and a second wire 47. The main driving unit 48 includes a first main driving unit 110, a second main driving unit 120, and a third main driving unit 130. Here, the first rotation member 41, the second rotation member 42, the third rotation member 43, and the fourth rotation member 44 of the first support driving unit 40 may be configured to be the same as the first embodiment and thus, a further detailed description related thereto will be omitted here.

The first wire 46 and the second wire 47 are connected to a pair of idler pulleys 40a and 40b, provided to each of the first rotation member 41, the second rotation member 42, the third rotation member 43, and the fourth rotation member 44, to be crossed in an “S” shape or an inverse “S” shape to thereby form a single closed loop wire.

The main driving unit 48 is connected to each of the first wire 46 and the second wire 47 to thereby adjust a position of the first wire 46 and the second wire 47, thereby enabling the entire wrist member 20 to perform a yaw motion by rotating the entire wrist member 20, enabling the plurality of effectors 30a and 30b to perform a pitch motion by simultaneously rotating the plurality of effectors 30a and 30b into a single direction, or enabling the plurality of effectors 30a and 30b to perform a forceps motion by rotating the plurality of effectors 30a and 30b into a direction increasing or decreasing a distance therebetween.

In the second embodiment of the present invention, the main driving unit 48 is twisted by 90 degrees with respect to either the plurality of effectors 30a and 30b or the wrist member 20 and thereby is mounted. That is, the main driving unit 48 is twisted by 90 degrees with respect to a shaft direction of the first pin 1 to be combined with the wrist member 20 to which the plurality of effectors 30a and 30b is mounted and a shaft direction of the first rotation shaft 110a of the first main driving unit 110 as shown in FIG. 9. The main driving unit 48 is mounted to be spaced apart from the shaft 10 and is connected to the plurality of effectors 30a and 30b through the first wire 46 and the second wire 47.

The main driving unit 48 that is mounted to be twisted with respect to the plurality of effectors 30a and 30b includes the first through third main driving unit s110, 120, and 130.

Since the main driving unit 48 is twisted with respect to the plurality of effectors 30a and 30b by a predetermined angle, for example, 90 degrees, and thereby is mounted, the first driving unit 110 may be connected to the first wire 46 and the second wire 47 to thereby enable the plurality of effectors 30a and 30b to perform the pitch motion by simultaneously rotating the plurality of effectors 30a and 30b into a single direction. Similar to the first embodiment of the present invention, such first driving unit 110 includes a pair of first rotation guide members 111, a pair of second rotation guide members 112, and a seesaw motion member 113.

The pair of first rotation guide members 111 are connected to the first wire 46, and are mounted on both sides of the seesaw motion member 113, respectively. The pair of second rotation guide members 112 are connected to the second wire 47, and are mounted on both sides of the seesaw motion member 113, respectively. The seesaw motion member 113 is mounted with the pair of first rotation guide members 111 and the pair of second rotation guide members 112 on both sides, respectively, and performs the seesaw motion by rotation of the first rotation shaft 114 to thereby adjust a position of the first wire 46 or the second wire 47 and enables the plurality of effectors 30a and 30b to perform the pitch motion by simultaneously rotating the plurality of effectors 30a and 30b into a single direction.

As another embodiment of the first main driving unit 110, as shown in FIG. 10, the first main driving unit 110 includes a pair of first rotation guide member 111, a pair of second rotation guide members 112, a pair of first support rotation guide members 111a, a pair of second support rotation members 111b, a pair of third support rotation guide members 112a, a pair of fourth support rotation guide members 112b, and a linear motion member 115.

The pair of first rotation guide members 111 are connected to the first wire 46, and the pair of second rotation guide members 112 are connected to the second wire 47. The pair of first support rotation guide members 111a are mounted on front of the pair of first rotation guide members 111, and are connected to the first wire 46. The pair of second support rotation guide members 111b are mounted on rear of the pair of first rotation guide members 111, and are connected to the first wire 46. The pair of third support rotation guide members 112a are mounted on front of the pair of second rotation guide members 112, and are connected to the second wire 47. The pair of four support rotation guide members 112b are mounted on rear of the pair of second rotation guide members 112 and are connected to the second wire 47. Each of the pair of first rotation guide members 111, the pair of second rotation guide members 112, the pair of first support rotation guide members 111a, the pair of second support rotation guide members 111b, the pair of third rotation support guide members 112a, and the pair of fourth rotation support guide members 112b is mounted to the linear motion member 115, and is applied with an idler pulley.

The linear motion member 115 is mounted with the pair of first rotation guide members 111 and the pair of second rotation guide members 112 on both sides, respectively, and performs a linear motion to thereby adjust a position of the first wire 46 or the second wire 47. The linear motion member 115 adjusting the position of the first and second wires 46 and 47 is connected to a linear motion mechanism (not shown) to perform the linear motion into indicator directions C5 and C6 by way of the linear motion mechanism. When the linear motion member 115 performs the linear motion into the indicator directors C5 and C6, a displacement corresponding to the linear motion may occur in the position of the first wire 46 or the second wire 47. Due to the displacement of wire position, the plurality of effectors 30a and 30b may perform the pitch motion based on the first pin 1 by simultaneously rotating into a single direction.

The second main driving unit 120 is mounted to be spaced apart from the first main driving unit 110, and is connected to the first wire 46 through the first main driving unit 110. The third main driving unit 130 is disposed to face the second main driving unit 120, and is connected to the second wire 47 through the first main driving unit 110. Since the support driving unit 40 is mounted to be twisted with respect to the plurality of effectors 30a and 30b, the second main driving unit 120 and the third main driving unit 130 rotate into the same direction to thereby enable the wrist member 20 to perform the yaw motion, or rotate into different directions to thereby enable the plurality of effectors 30a and 30b to perform the forceps motion of rotating into a direction increasing or decreasing a distance therebetween. An amount of the forceps motion may be determined based on a difference of angle of rotation between the second main driving unit 120 and the third main driving unit 130.

The second main driving unit 120 and the third main driving unit 130 include second rotation shafts 122 and 132, and pulleys 121 and 131, respectively.

Each of the second rotation shafts 122 and 132 is mounted on front or rear of the first main driving unit 110, and is connected to a driving source (not shown). The second rotation shafts 122 and 132 connected to the driving source rotate into the indicator directions C1 and C2 by operation of the driving source. The pulleys 121 and 131 are connected to the second rotation shafts 122 and 132, and rotate by rotation of the second rotation shafts 122 and 132, respectively, to thereby adjust a position of the first wire 46 or the second wire 47. That is, the pulleys 121 and 131 rotating by the second rotation shafts 122 and 132 that rotate by the driving source may adjust the position, for example, length of the first wire 46 or the second wire 47, thereby enabling the yaw motion of the wrist member 20 or the forceps motion of the plurality of effectors 30a and 30b.

For example, when the pulleys 121 and 131, respectively provided to the second main driving unit 120 and the third main driving unit 130, rotate into the same direction, the entire wrist member 20 may rotate to thereby perform the yaw motion. On the contrary, when the pulleys 121 and 131 rotate into different directions, the plurality of effectors 30a and 30b may rotate into a direction increasing or decreasing a distance therebetween and thereby perform the forceps motion.

Hereinafter, an operation effect of the surgical instrument according to the first embodiment of the present invention constructed as above will be described with reference to FIGS. 2 through 5 and FIGS. 6 and 7.

Initially, a pitch motion of the surgical instrument of the present invention will be described.

The first wire 46 may be moved into the indicator direction F2 by rotation of the second main driving unit 120 whereby a position of the first wire 46 may be adjusted. When the position of the first wire 46 is adjusted, one idler pulley 40a provided to each of the first through fourth rotation member 41, 42, 43, and 44 connected to the first wire 46, and one capstan 31 among the plurality of effectors 30a and 30b may rotate. When one capstan 31 rotates into an indicator direction B based on the third pin 3, one end effector 34 among the plurality of effectors 30a and 30b may perform the pitch motion into an indicator direction B2.

The second wire 47 may be moved into the indicator direction F2 by rotation of the third main driving unit 130 whereby a position of the second wire 47 may be adjusted. When the position of the second wire 47 is adjusted, another idler pulley 40b, provided to each of the first through fourth rotation members 41, 42, 43, and 44 connected to the second wire 47, and another capstan 31 among the plurality of effectors 30a and 30b may rotate. When the other capstan 31 rotates into the indicator direction B based on the third pin 3, the other end effector 34 among the plurality of effectors 30a and 30b may perform the pitch motion into an indicator direction B3 of FIG. 7.

When the second main driving unit 120 and the third main driving unit 130 rotate into the same direction, for example, the indicator direction C1 or C2, each of the first wire 46 and the second wire 47 may be moved into the indicator direction F2. When each of the first wire 46 and the second wire 47 is moved into the indicator direction F2, the plurality of idler pulleys 40a and 40b, provided to each of the first through fourth rotation member 41, 42, 43, and 44 connected to the first wire 46 and the second wire 47, and the capstan 31 provided to each of the plurality of effectors 30a and 30b may rotate. When the capstan 31 rotates into the indicator direction B based on the third pin 3, the end effector 34 provided to each of the plurality of effectors 30a and 30b may perform the pitch motion into the indicator direction B3 of FIG. 7.

Hereinafter, a yaw motion of the surgical instrument of the present invention will be described.

The seesaw motion member 113 may rotate by the first rotation shaft 114 connected by a driving source (not shown). When the seesaw motion member 113 rotates by an angle of rotation α (see FIG. 6), one of the first wire 46 and the second wire 47 may rotate into the indicator direction F1 by the angle of rotation α of the seesaw motion member 113 to thereby adjust a wire position. When the wire position is adjusted, the wrist member 20 may rotate into an indicator direction A based on the first pin 1 to thereby perform the yaw motion. When the wrist member 20 performs the yaw motion, the first rotation member 41 and the second rotation member 42 may not rotate and be fixed to the second pin 2 and perform only a guide functionality. In this state, the third rotation member 43 and the fourth rotation member 44 may rotate into the indicator direction A based on the first pin 1, and the wrist member 20 may perform the yaw motion as shown in FIG. 6 by the above rotation. That is, when the wrist member 20 performs the yaw motion, the first rotation member 41 and the second rotation member 42 may not rotate and be fixed to the second pin 2 and thereby perform only a guide functionality. The third rotation member 43 and the fourth rotation member 44 enable the wrist member 20 to perform the yaw motion by employing the third rotation member 43 and the fourth rotation member 44 as a wrist joint W (see FIG. 2).

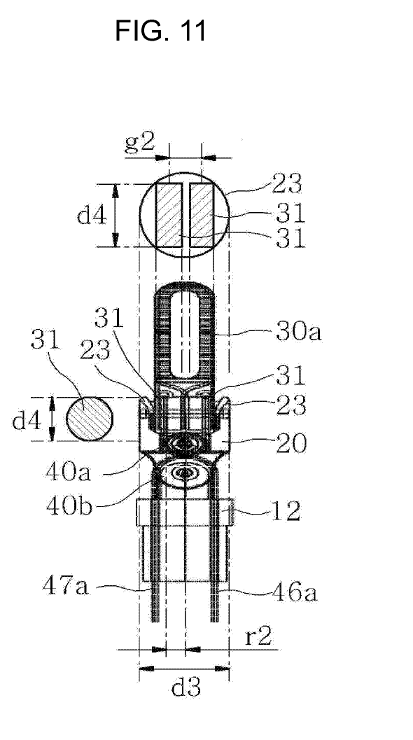

As described above, when the third rotation member 43 and the fourth rotation member 44 are employed as the wrist joint W (see FIG. 2), the first rotation member 41 and the second rotation member 42 perform only the guide functionality in the case of adjusting the position, for example, length of the first wire 46 and the second wire 47 and thus, the radius r2 (see FIG. 7) of the first rotation member 41 and the second rotation member 42 may be formed to be small. When the radius r2 of the first rotation member 41 and the second rotation member 42 is formed to be small, the distance g2 (see FIG. 11) between the capstans 31 may also decrease. When the distance g2 between the capstans 31 decreases, a diameter d4 (see FIG. 11) of the capstan 31 may be formed to be large. Accordingly, it is possible to drive the pitch motion of the end effector 34 using further small torque.

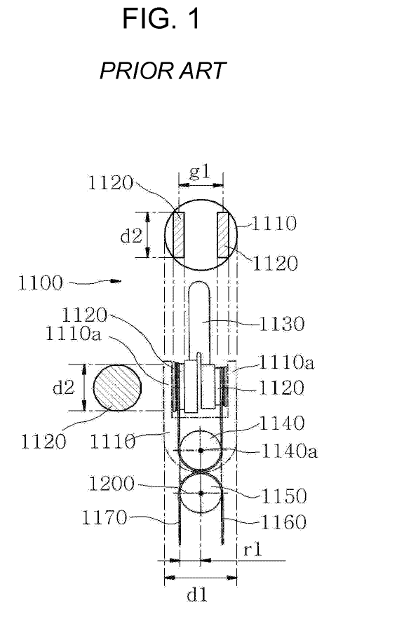

That is, when the diameter d3 of the surgical instrument of the present invention is identical to the diameter d1 (see FIG. 1) of the conventional surgical instrument, and when decreasing the radius r2 of a pair of first and second rotation members 41 and 42, the surgical instrument of the present invention may drive the end effectors 34 with relatively small torque compared to the conventional surgical instrument and thus, enables a minute control.

In addition, the surgical instrument of the present invention may drive the end effectors 34 with further small torque and thus, tension applied to the first wire 46 and the second wire 47 may decrease. Accordingly, it is possible to enhance the durability and thereby enhance reliability of the surgical instrument.

Similar to the operation effect of the surgical instrument according to the first embodiment of the present invention constructed as above, an operation of the surgical instrument according to the second embodiment of the present invention will be described in the following with reference to FIG. 10.

The main driving unit 48 is mounted to be twisted with respect to the plurality of effectors 30a and 30b by 90 degrees, the first main driving unit 110 may simultaneously rotate the plurality of effectors 30a and 30b into a single direction by moving a pair of third support rotation guide members 112a and a pair of fourth support rotation guide members 112b that are disposed on top and bottom of one side of the linear motion member 115, and thereby enable the plurality of effectors 30a and 30b to perform the pitch motion.

Conversely, the first main driving unit 110 may simultaneously rotate the plurality of effectors 30a and 30b into different directions by moving a pair of first support rotation guide members 111a and a pair of second support rotation guide members 111b that are disposed on top and bottom of another side of the linear motion member 115, and thereby enable the plurality of effectors 30a and 30b to perform the pitch motion. Also, when the second main driving unit 120 and the third main driving unit 130 rotate into the same direction due to the support driving unit 40 that is mounted to be twisted with respect to the plurality of effectors 30a and 30b, the wrist member 20 may perform the yaw motion. Conversely, when the second main driving unit 120 and the third main driving unit 130 rotate into the directions, the plurality of effectors 30a and 30b may perform the forceps motion into a direction increasing or decrease a distance therebetween.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

1. A surgical instrument, comprising:
a shaft;
a wrist member comprising a guide member, a wrist body connected to the guide member and formed with a plurality of installation holes, and a plurality of protruding members each disposed to face the wrist body and having an insertion groove, and connected to the shaft;
a plurality of effectors each comprising a capstan, a protruding member integrally formed with the capstan and formed with an insertion hole, a fixing member inserted into the insertion hole of the protruding member, and an end effector formed on the capstan to perform a pitch motion by rotation of the capstan, and connected to the wrist member by a pin;
a support driving unit having first through fourth rotation members inserted into pins that are mounted on the wrist member, respectively, and each comprising a pair of idler pulleys, and first and second wires connected to the plurality of effectors and the first through fourth rotation members, respectively;
a main driving unit connected to the support driving unit by wire and having at least one driving unit,
wherein the main driving unit enables the entire wrist member to perform a yaw motion by rotating the entire wrist member, and enables the plurality of effectors to perform a pitch motion by simultaneously rotating the plurality of effectors into a single direction, and enables the plurality of effectors to perform a forceps motion of rotating into a direction increasing or decreasing a distance between the plurality of effectors.

2. The surgical instrument of claim 1, wherein the shaft comprises:
a hollow member;
a support member mounted on the hollow member and formed with a plurality of guide holes; and
a plurality of protruding members disposed to face the support member and each formed with an insertion hole in which a pin is to be inserted,
wherein an inclined surface is formed between the support member and each of the plurality of protruding members.

3. The surgical instrument of claim 1, wherein the wrist body of the wrist member further comprises a plurality of insertion holes that is connected to the plurality of installation holes and in which a pin is to be inserted, and each of the plurality of insertion holes is formed to be inclined.

4. The surgical instrument of claim 1, wherein a diameter of the first and second rotation members of the support driving unit is formed to be less than a diameter of the third and fourth rotation members.

5. The surgical instrument of claim 1, wherein a radius of the first and second rotation members of the support driving unit is formed to be 0.3 to 0.9 folds of a radius of the third and fourth rotation members.

6. The surgical instrument of claim 1, wherein, between the first and second wires of the support driving unit, the first wire is connected to one capstan among the plurality of effectors and one idler pulley among the first through fourth rotation members to be crossed in an “S” shape or an inverse “S” shape, and the second wire is connected to another capstan among the plurality of effectors and another idler pulley to be crossed in the “S” shape or the inverse “S” shape.

7. The surgical instrument of claim 1, wherein each of the first and second wires of the support driving unit is fixed to the capstan by the fixing member of an effector in the case of being connected to each capstan, and is inserted and thereby mounted to be guided and thereby moved by a plurality of guide holes formed in a support member of the shaft.

8. The surgical instrument of claim 1, wherein the main driving unit comprises:
a first main driving unit spaced apart from the shaft to enable the wrist member to perform the yaw motion based on a pin;
a second main driving unit mounted on front or rear of the first main driving unit and connected to a wire through the first main driving unit to enable one of the plurality of effectors to perform the pitch motion based on the pin; and
a third main driving unit disposed to face the second main driving unit and connected to the wire through the first main driving unit to enable another one of the plurality of effectors to perform the pitch motion based on the pin.

9. The surgical instrument of claim 8, wherein the first main driving unit comprises:
a pair of first rotation guide members connected to the first wire;
a pair of second rotation guide members connected to the second wire; and
a seesaw motion member mounted on both sides of each of the pair of first rotation guide members and the pair of second rotation guide members to perform a seesaw motion by rotation of a first rotation shaft, and enabling the wrist member to perform the yaw motion based on a first pin by adjusting a position of the first wire and the second wire.

10. The surgical instrument of claim 8, wherein the first main driving unit comprises:
a pair of first rotation guide members connected to the first wire;
a pair of second rotation guide members connected to the second wire;
a pair of first support rotation guide members mounted on front of the pair of first rotation guide members and connected to the first wire;
a pair of second support rotation guide members mounted on rear of the pair of first rotation guide members and connected to the first wire;
a pair of third support rotation guide members mounted on front of the pair of second rotation guide members and connected to the second wire;
a pair of fourth support rotation guide members mounted on rear of the pair of pair of second rotation guide members and connected to the second wire; and
a linear motion member mounted with the pair of first rotation guide members and the pair of second rotation guide members on both sides of the linear motion member, respectively, to perform a linear motion, and enabling the wrist member to perform the yaw motion based on a first pin by adjusting a position of the first wire and the second wire.

11. The surgical instrument of claim 10, wherein the idler pulley is applied to each of the pair of first and second rotation guide members and the pair of first through fourth support rotation guide members of the first main driving unit.

12. The surgical instrument of claim 8, wherein each of the first and second main driving units comprises:
a second rotation shaft mounted on front or rear of a first wire driving source; and
a pulley connected to the second rotation shaft and rotating by rotation of the second rotation shaft to adjust a position of the first wire or the second wire, and
the pulley is mounted so that the pair of first rotation guide members and the pair of second rotation guide members cross each other.

13. The surgical instrument of claim 12, wherein when the second rotation shaft provided to each of the first and second main driving units rotates in the same direction, each of the first and second main driving units enables the plurality of effectors to perform the pitch motion by simultaneously rotating the plurality of effectors into a single direction, and when the second rotation shaft rotates into a different direction, each of the first and second main driving units enables the plurality of effectors to perform the forceps motion of rotating into a direction increasing or decreasing a distance between the plurality of effectors.

14. A surgical instrument, comprising:
a shaft;
a wrist member comprising a guide member, a wrist body connected to the guide member and formed with a plurality of installation holes, and a plurality of protruding members each disposed to face the wrist body and having an insertion groove, and connected to the shaft;
a plurality of effectors each connected to the wrist member by a pin;
a support driving unit having first through fourth rotation members inserted into pins that are mounted on the wrist member, respectively, and first and second wires sequentially connected to the plurality of effectors and the first through fourth rotation members, respectively; and
a main driving unit comprising a first main driving unit connected to the first and second wires to enable the plurality of effectors to perform a pitch motion by simultaneously rotating the plurality of effectors into a single direction, a second main driving unit mounted on front or rear of the first main driving unit and connected to the first wire through the first main driving unit, and a third main driving unit disposed to face the second main driving unit and connected to the second wire through the first main driving unit, and the main driving unit connected to the support driving unit using a wire and mounted to be twisted at a predetermined angle with respect to the plurality of effectors,
wherein the second main driving unit and the third main driving unit of the main driving unit rotate into the same direction to thereby enable the entire wrist member to perform a yaw motion by rotating the entire wrist member, or rotate into different directions to thereby enable the plurality of effectors to perform a forceps motion of rotating into a direction increasing or decreasing a distance between the plurality of effectors.

15. The surgical instrument of claim 14, wherein a radius of the first and second rotation members of the support driving unit is formed to be 0.3 to 0.9 folds of a radius of the third and fourth rotation members.

16. The surgical instrument of claim 14, wherein the first and second wires of the support driving unit is formed as a single closed loop wire by the main driving unit twisted and thereby mounted with respect to the effectors.

17. The surgical instrument of claim 14, wherein the main driving unit is twisted by 90 degrees with respect to the plurality of effectors and thereby is mounted.

18. The surgical instrument of claim 14, wherein the first main driving unit comprises:
a pair of first rotation guide members connected to the first wire;
a pair of second rotation guide members connected to the second wire; and
a seesaw motion member mounted on both sides of each of the pair of first rotation guide members and the pair of second rotation guide members to perform a seesaw motion by rotation of a first rotation shaft, and enabling the plurality of effectors to perform the pitch motion by adjusting a loop position of the first wire and the second wire to thereby simultaneously rotate the plurality of effectors into a single direction.

19. The surgical instrument of claim 14, wherein the first main driving unit comprises:
a pair of first rotation guide members connected to the first wire;
a pair of second rotation guide members connected to the second wire;
a pair of first support rotation guide members mounted on front of the pair of first rotation guide members and connected to the first wire;
a pair of second support rotation guide members mounted on rear of the pair of first rotation guide members and connected to the first wire;
a pair of third support rotation guide members mounted on front of the pair of second rotation guide members and connected to the second wire;
a pair of fourth support rotation guide members mounted on rear of the pair of pair of second rotation guide members and connected to the second wire; and
a linear motion member mounted with the pair of first rotation guide members and the pair of second rotation guide members on both sides of the linear motion member, respectively, to perform a linear motion, and enabling the plurality of effectors to perform the pitch motion by adjusting a loop position of the first wire and the second wire to thereby simultaneously rotate the plurality of effectors into a single direction.

20. The surgical instrument of claim 14, wherein each of the second and third main driving units comprises:
a second rotation shaft mounted on front or rear of a first wire driving source; and
a pulley connected to the second rotation shaft and rotating by rotation of the second rotation shaft to adjust a position of the first wire or the second wire, and
the pulley is mounted so that the pair of first rotation guide members and the pair of second rotation guide members cross each other.

21. The surgical instrument of claim 14, wherein each of the second and third main driving units is provided with a pulley, and is configured to enable the entire wrist member to perform the yaw motion by rotating the entire wrist member when the pulleys rotate into the same direction, and to enable the plurality of effectors to perform the forceps motion of rotating into a direction increasing or decreasing a distance between the plurality of effectors when the pulleys rotate into different directions.

22. A surgical instrument, comprising:
a plurality of effectors;
a wrist member comprising a guide member, a wrist body connected to the guide member and formed with a plurality of installation holes, and a plurality of protruding members each disposed to face the wrist body and having an insertion groove, and connected to the effector;
a shaft comprising a hollow member, a support member connected to the hollow member, and a protruding member formed on each of both sides of the support member, and connected to the wrist member;
a support driving unit comprising a pair of rotation members and at least one wire; and
a main driving unit having a plurality of driving units for operating the at least one wire of the support driving unit,
wherein the main driving unit comprises a first main driving unit connected to the first and second wires to enable the plurality of effectors to perform a pitch motion by simultaneously rotating the plurality of effectors into a single direction, a second main driving unit mounted on front or rear of the first main driving unit and connected to the first wire through the first main driving unit, and a third main driving unit disposed to face the second main driving unit and connected to the second wire through the first main driving unit, and the main driving unit is connected to the support driving unit using a wire and mounted to be twisted at a predetermined angle with respect to the plurality of effectors,
the main driving unit comprises a pair of first and second rotation guide members connected to the first and second wires, respectively, a pair of first through fourth support rotation guide members mounted on front and rear of the first and second rotation guide members, respectively, and a linear motion member mounted on each of both sides of each of the first and second rotation guide members to perform a linear motion, and enabling the plurality of effectors to perform the pitch motion by adjusting a loop position of the first and second wires and thereby simultaneously rotating the plurality of effectors into a single direction,
each of the second and third main driving units comprises a second rotation shaft mounted on front or rear of the first main driving unit and a pulley connected to the second rotation shaft and rotating by rotation of the second rotation shaft to adjust a position of the first wire or the second wire, and
a diameter of the first and second rotation members of the driving unit is formed to be less than a diameter of the third and fourth rotation members in order to decrease a diameter of a capstan.

23. A surgical instrument, comprising:
a plurality of effectors each comprising a capstan, a protruding member integrally formed with the capstan and formed with an insertion hole, and a fixing member inserted into the insertion hole of the protruding member;
a wrist member comprising a guide member, a wrist body connected to the guide member and formed with a plurality of installation holes, and a plurality of protruding members each disposed to face the wrist body and having an insertion groove, and connected to the effector;
a shaft comprising a hollow member, a support member connected to the hollow member, and a protruding member formed on each of both sides of the support member, and connected to the wrist member;
a support driving unit comprising a pair of plural rotation members and at least one wire; and
a main driving unit having a plurality of driving units for operating the at least one wire of the support driving unit,
wherein the main driving unit comprises a first main driving unit connected to the first and second wires to enable the plurality of effectors to perform a pitch motion by simultaneously rotating the plurality of effectors into a single direction, a second main driving unit mounted on front or rear of the first main driving unit and connected to the first wire through the first main driving unit, and a third main driving unit disposed to face the second main driving unit and connected to the second wire through the first main driving unit, and the main driving unit is connected to the support driving unit,
the main driving unit comprises a pair of first rotation guide members connected to the first wire, a pair of second rotation guide members connected to the second wire, and a seesaw motion member mounted on both sides of each of the pair of first rotation guide members and the pair of second rotation guide members to perform a seesaw motion by rotation of a first rotation shaft, and enabling the plurality of effectors to perform the pitch motion by adjusting a loop position of the first wire and the second wire to thereby simultaneously rotate the plurality of effectors into a single direction,
and the main driving unit is twisted by 90 degrees with respect to either the plurality of effectors or the wrist member and thereby is mounted.
